(19) United States
(12) Patent Application Publication
(10) Pub. No.: US 2010/0316736 A1
(43) Pub. Date: Dec. 16, 2010

(54) APPETITE SUPPRESSANT

(75) Inventor: Refael Aharon, Jerusalem (IL)

Correspondence Address: THE NATH LAW GROUP, 112 South West Street, Alexandria, VA, 22314, US

(73) Assignee: DESERT LABS AGRICULTURE COOPERATIVE ASSOCIATION LTD., Eilot (IL)

(21) Appl. No.: 12/739,264

(22) PCT Filed: Oct. 23, 2008
(86) PCT No.: PCT/IL2008/001402

§ 371 Date: Apr. 22, 2010

Related U.S. Application Data
(60) Provisional application No. 60/996,005, filed on Oct. 24, 2007.

(51) Int. Cl. A61K 36/27; A61P 3/04

(52) U.S. Cl. 424/725

(57) ABSTRACT

Provided is an extract of Hoodia Parviflora, as well as its use as an appetite suppressant.

### FIELD OF THE INVENTION

This invention relates to the field of appetite suppressants from Hoodia plant.

### BACKGROUND OF THE INVENTION

Obesity is a major public health concern because of its increasing prevalence and associated health risks, including coronary heart disease, strokes, hypertension, type 2 diabetes mellitus, dyslipidemia, sleep apnea, osteoarthritis, gall bladder disease, depression, and certain forms of cancer (e.g., endometrial, breast, prostate, and colon).

Hoodia, from the Asclepiadaceae subfamily in the Apocynaceae family, is a succulent plant from Southern Africa that contains substances which suppress hunger, appetite, and thirst. The use of certain species of Hoodia as an appetite suppressant is supported by colorful folklore history and recent scientific studies. Tribesmen hunters in Africa have used Hoodia for many years to prevent hunger on long hunting trips.

The (active) ingredient of Hoodia which appears to be responsible for the appetite suppressing effect is named P57, an oxypregnane steroidal glycoside with the chemical name (3β,12β,14β)-3-[(O-6-Deoxy-3-O-methyl-β-D-glucopyrano-(1→4)-O-2,6-dideoxy-3-O-methyl-β-D-ribo hexopyranosyl-(1→4)-2,6-dideoxy-3-O-methyl-β-D-ribo-hexopyranosyl)oxy]-14-hydroxy-12-[[(2E)-2-methyl-1-oxo-2-butenyl]oxy]pregn-5-en-20-one.

Within the genus Hoodia, mostly the species Hoodia gordinnii is used as a natural appetite suppressant.

### SUMMARY OF THE INVENTION

The subject invention now provides an extract of Hoodia Parviflora, the appetite suppressant activity of which is more efficient than those of other Hoodia species.

### BRIEF DESCRIPTION OF THE FIGURES

FIG. 1 shows food consumption in male and female rats after 5 and 14 days of treatment with several species of Hoodia;

FIG. 2 shows body weight in female rats after administration of several types of Hoodia after different intervals of time; and

FIG. 3 shows the P57 content in several species of Hoodia.

### DETAILED DESCRIPTION OF THE INVENTION

The subject invention provides an extract of Hoodia parviflora.

Hoodia as used herein includes any species of Hoodia, such as, but not limited to, Hoodia parviflora, Hoodia gordonii, Hoodia macrantha, Hoodia currorii, Hoodia lugardii, Hoodia ruschii, Hoodia tugardii and mixtures thereof.

An extract as used herein includes, but is not limited to, liquid extracts, solid extracts or spray-dried extracts, e.g. sap and plant solids. A liquid extract may be prepared by homogenizing the whole plant. Sap may be harvested from the plant through a wound or opening. An extract as used herein may be purified, partially purified, concentrated and/or fractionated.

A composition comprising an extract of the invention as used herein can be, but is not limited to, a nutritional composition, a nutraceutical composition, a pharmaceutical composition, a functional food and so forth. A composition as used herein can be edible (e.g. it can be eaten, drunk, swallowed and so forth). A composition as used herein may be in liquid form or solid form, such as, but not limited to, an oil, a beverage, a tablet, a powder, an ice cream, a frozen tablet, a milk additive, and so forth.

As used herein, the term “nutritional” denotes that which is beneficially assimilated by the subject for normal growth, general health or vitality of the subject.

The term “edible” as used herein does not necessarily imply a compound which is palatable, or even one which can safely, as proven by animal experiments, be eaten, in its pure form. Rather, the term is intended to mean that the compound must be one which is acceptable for use in a food at the concentrations in which it is used in the composition as determined by authorities such as the FDA.

A nutritional composition as used herein is intended to encompass supplementation by enteral method and includes, without limitation, dietary supplements and/or medicinal supplements, in liquid or solid form, e.g., as a natural extract, a beverage, a (frozen) tablet, or a powder, and so forth. By way of example, the nutritional composition of the invention is easily administered to a mammal by enteral administration, preferably by oral administration as a food product or a food additive. The food product can be a solid or a liquid. Alternatively, the nutritional supplement is in a form conventionally known to those skilled in the art as suitable for administering oil and water-based vitamins and metabolites, such as, without limitation, encapsulation within a soft gelatin capsule.

As used herein, the term “dietary” denotes that which is naturally a part of the subject's diet.

A nutraceutical composition as used herein can be any substance that may be considered a food or part of a food and provides medical or health benefits, including the prevention and treatment of obesity. Such nutraceutical compositions include, but are not limited to, a food additive, a food supplement, a dietary supplement, a vegetable, a herbal product, and a processed food such as cereal, soup, beverage and stimulant functional food and pharmafood.

A functional food as used herein can be any functional food, including, but not limited to, dairy product, ice-cream, biscuit, soy product, bakery, pastry and bread, sauce, soup, prepared food, frozen food, condiment, confectionary, oils and fat, margarine, spread, filling, cereal, instant product, drinks and shake, infant food, bar, snack, candy and chocolate product.

As used herein, the term “food additive” denotes a substantially-pure material which is added to food for a nutritional benefit.

As used herein, the term “food product” denotes a product intended for ingestion by a mammal, including humans, which has nutritional value.

A plant as used herein means any part of the plant, such as, but not limited to stems, arms, leaves and mixtures thereof.

Hoodia Parviflora extracts of the invention are useful as appetite suppressants. An appetite suppressant as used herein denotes activity which tends to limit appetite and/or increase the sense of satiety, and thus tends to reduce total calorific intake; which in turn tends to counteract obesity. Accordingly, this invention extends to a method of treating or preventing obesity in a human or non-human animal which comprises administering to said human or non-human animal an obesity treating or preventing amount of Hoodia extract of the invention.

As used herein, the term “subject” expressly includes human and non-human mammalian subjects.

The term “non-human animal” as used herein extends to, but is not restricted to, companion animals, e.g. household pets and domesticated animals. Non-limiting examples of such animals include cattle, sheep, ferrets, swine, camels, horses, poultry, fish, rabbits, goats, dogs and cats.

The present invention also relates to pharmaceutical compositions. A pharmaceutical composition as used herein means a composition comprising an extract of the invention, in admixture with acceptable auxiliaries such as, but not limited to, pharmaceutically acceptable carriers, diluents, adjuvants, excipients, or vehicles, such as preserving agents, fillers, disintegrating agents, wetting agents, emulsifying agents, suspending agents, sweetening agents, flavouring agents, perfuming agents, antibacterial agents, antifungal agents, lubricating agents and dispensing agents, depending on the nature of the mode of administration and dosage forms.

The auxiliaries must be “acceptable” in the sense of being compatible with the other ingredients of the composition and not deleterious to the recipients thereof, i.e. pharmaceutically acceptable.

“Pharmaceutically acceptable” means it is, within the scope of sound medical judgement, suitable for use in contact with the cells of humans and animals without undue toxicity, irritation, allergic response and the like, and are commensurate with a reasonable benefit/risk ratio.

Suitable routes of administration for the extracts of the subject invention are enteral, topical, transdermal, oral, buccal and sublingual and so forth.

“Pharmaceutically acceptable dosage forms” as used herein include, but are not limited to dosage forms such as tablets, dragees, powders, elixirs, syrups, liquid preparations, including suspensions, sprays, lozenges, emulsions, solutions, granules and capsules, including liposome preparations. The active ingredient may also be presented as a bolus or paste. Techniques and formulations generally may be found in Remington, Pharmaceutical Sciences, Mack Publishing Co., Easton, Pa., latest edition.

Extracts of the invention and compositions comprising such extracts may be administered under the supervision of a medical specialist, or may be self-administered.

The exact dose and regimen of administration of an extract of the invention or a composition comprising such extract will necessarily be dependent upon the effect to be achieved (e.g. appetite suppression, treatment of obesity) and may vary with the route of administration, and the age and condition of the individual subject to whom the extract is to be administered.

A dosage for humans is likely to contain from about 10 to about 1000 mg (dry weight) per 70 kg body weight per day. The desired dose may be presented as one dose or as multiple sub-doses administered at appropriate intervals.

The compositions may be prepared by any method well known in the art of pharmacy.

Such methods include the step of bringing in association a Hoodia Parviflora extract of the invention with any auxiliary agent.

The invention further includes a composition comprising an extract of the invention, in combination with packaging material, including instructions for the use of the composition for a use as hereinbefore described.

Extracts of the invention may be administered in conjunction with other ingredients, including, but not limited to folic acid, vitamins, minerals, anti-oxidants, other extracts from plants or fruit, liquid flavors and so forth.

Liquid flavors as used herein means any liquid flavor characterized by low viscosity.

Vitamins as used herein means any vitamin such as, but not limited to, B1, B2, B3, B6, B12, Folic Acid, Vitamin C, Biotin, Pantothenic acid, K, A, D, E and so forth.

Antioxidants as used herein are meant to encompass any antioxidant such as, but not limited to a compound that has antioxidant activity.

Minerals as used herein means any mineral such as, but not limited to, Na, K, Cl, Ca, P, Mg, Fe, I, Cu, Zn, Mn, Fl, and so forth.

Thus, the subject invention provides an extract of Hoodia Parviflora. In one embodiment, the extract is liquid. In another embodiment the extract is solid.

The subject invention further provides a composition comprising an extract of the invention.

In one embodiment, the subject invention provides a composition containing an appetite suppressant effective amount of an extract of the invention.

It is envisaged that a composition of the invention can be a pharmaceutical composition.

It is further envisaged that a composition of the invention is edible. In one embodiment, such an edible composition is selected from the group consisting of a dietary supplement, a nutraceutical, a food additive, a food product, or a beverage. In a specific embodiment, the food product is an ice-cream or a frozen-ice.

The subject invention further provides a frozen tablet containing an extract of the invention.

One aspect of the invention is a use of a composition of the invention for the manufacture of an appetite suppressant. Another aspect of the invention is a use of a composition of the invention for the manufacture of a medicament for treating or preventing obesity.

The subject invention further provides a use of a composition of the invention for suppressing appetite. The subject invention further provides a use of a composition of the invention for the treatment or prevention of obesity.

The subject invention further encompasses a method of treating or preventing obesity comprising administering a composition of the invention to a subject, suffering from, or at risk for, obesity.

The subject invention also encompasses a method of suppressing appetite in a subject comprising administering a composition of the invention to the subject. In one embodiment, the subject is a human or a non-human animal.

The subject invention further provides a food additive comprising an extract of the invention. The subject invention also provides a use of such food additive for the manufacture of a composition for suppressing appetite and a use of such food additive for suppressing appetite.

The subject invention further provides ice cream comprising an extract of the invention. In one embodiment, the ice-cream is a popsickle.

The invention is further described in the following examples, which are not in any way intended to limit the scope of the inventions as claimed

### EXAMPLES

### Example 1

### Preparation of an Extract of Hoodia Parviflora

A Hoodia ParviFlora extract was prepared as follows:

Fresh Hoodia parviflora plants were washed in water and disinfectant. The washed plants were frozen and, cut to size of 0.1-10 cm², and water was added to the cut Hoodia plant tissue thereby obtaining suspended Hoodia parviflora. The suspended Hoodia parviflora plant tissue was further disintegrated and homogenized for 30 minutes in an ultrasonic bath filled with water at 0-10° C. Liquid Hoodia filtrate was separated from solid Hoodia sediment by centrifuging or filtering, thereby obtaining a liquid Hoodia extract and a solid Hoodia extract.

### Example 2

60 healthy young adult Sprague-Dawley rats were housed under standard laboratory conditions, air conditioned and filtered (HEPA F6/6) with adequate fresh air supply (31 air changes/hour). The rats were kept in a climate controlled environment. Temperatures ranged between 20-24° C. and relative humidity (RH) was between 30-70% with 12 hours light and 12 hours dark cycle.

The rats were randomly divided into 10 groups, each group having 3 male and 3 female rats. Starting body weight of male rates ranges from 225-250 gram and of female rates from 150-170 grams.

Group 1 received 1 ml oral placebo (hereinafter “C”) for 10 days.

Group 2 received orally 1 ml saline (0.9% NaCl) containing, 6.5 mg of 7-year old Hoodia Gordonii (hereinafter “X”) for 10 days

Group 3 received orally 1 ml saline (0.9% NaCl) containing 6.5 mg of 5 year old Hoodia big Gordonii (hereinafter “B”) for 10 days.

Group 4 received orally 1 ml saline (0.9% NaCl) containing 6.5 mg of 2-year old Hoodia Yotvata Gordonii (hereinafter “GT”) for 10 days.

Group 5 received orally 1 ml saline (0.9% NaCl) containing 6.5 mg of 5 year old Hoodia Parviflora (hereinafter “P”) for 10 days.

Group 6 received orally 1 ml saline (0.9% NaCl) containing 6.5 mg of 2-year old Hoodia Rushi (hereinafter “R”) for 10 days.

Group 7 received orally 1 ml saline (0.9% NaCl) containing 6.5 mg of 2 year old Hoodia macarenta (hereinafter “M”) for 10 days.

Group 8 received orally 1 ml saline (0.9% NaCl) containing 13 mg 5 year old Hoodia Parviflora (hereinafter “H”) for 10 days.

Group 9 received orally 1 ml saline (0.9% NaCl) containing 13 mg 7 year old Hoodia Gordonii (hereinafter “S”) for 10 days.

Group 10 received orally 1 ml saline (0.9% NaCl) containing 13 mg of 2 year old Hoodia Gordonii (hereinafter “Y”) for 10 days.

All Hoodia extracts were prepared as described in Example 1. The liquid extracts were stored in frozen form at −20° C. and dissoluted in 1.3 ml saline prior to use, 1 ml of which was administered.

The rats were further fed “free feeding” ad libitum, commercial rodent diet (Teklad Certified Global 18% Protein Diet cat#: 106S8216). The rats further had free access to drinking water obtained from the municipal supply. Reasonably expected contaminants in food and water supplies should not have the potential to influence the outcomes of the experiment.

Body weights were measured on days-1, 3, 5, 8, 9, 10, 11, 12 and 14.

Clinical signs were measured after 0.5, 1, 2 and 6 hours on the first day of dosing and then once daily.

Morbidity and mortality was evaluated twice a day.

Food consumption was measured on days-1, 5, 8, 11 and 14.

Water consumption was measured on days-1, 5, 8, 11 and 14.

Final bleeding and serum preparation took place on day 15, the last day of the study: the animals were sacrificed by Ketamine/Xylazine (85 mg/kg and 5 mg/kg respectively) and opening of the thoracic cavity followed by final bleeding. Gross pathology was performed examining the major tissue and organ systems: Brain; Lymph nodes (cervical, mandibular); Salivary glands; Lungs; Thyroid (Parathyroid); Thymus; Heart; Esophagus; Stomach; Pancreas; Duodenum; Jejunum; Ileum; Cecum; Colon; Liver; Kidneys; Adrenals; Spleen; Bladder; Testes; and Ovaries.

Liver from each animal was collected and immediately frozen at −70° C. Some of the livers were sent for histopathological evaluation.

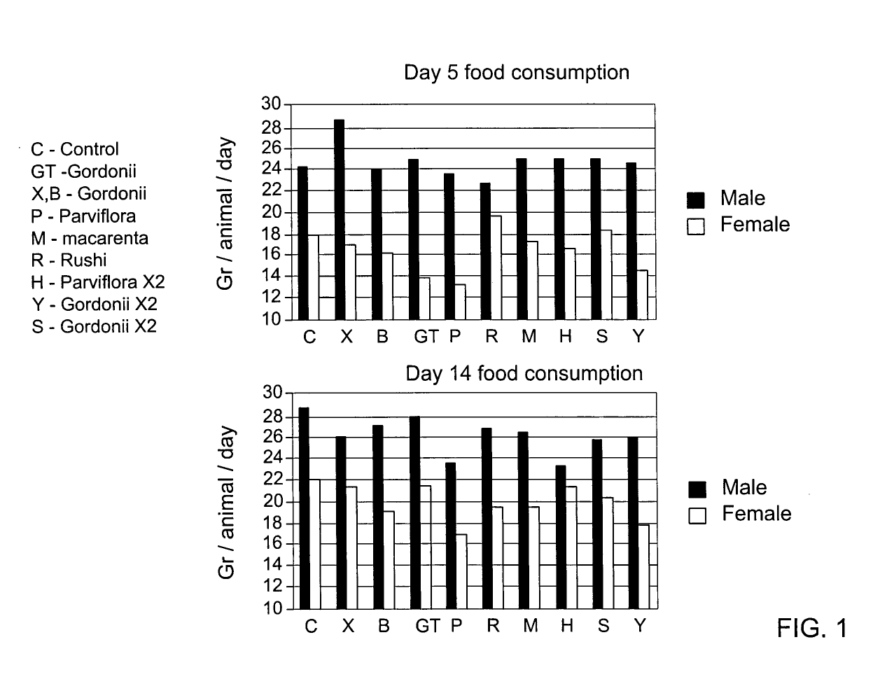

FIG. 1 shows that food consumption in female rats after 5 and 14 days of treatment with the extract of Hoodia parviflora at a daily dose level of 6.5 mg/rat (Group P) was lower compared to the food consumption of female rats who received other species of Hoodia. FIG. 1 also shows that in female rats this effect is not maintained at a double dose (13 mg/rat-Group H).

FIG. 1 further shows that food consumption in male rats after 14 days of treatment with the extract of Hoodia parviflora at daily dose levels of both 6.5 mg/rat and 13 mg/rat (Groups P and H respectively) was lower compared to the food consumption of male rats who received other species of Hoodia.

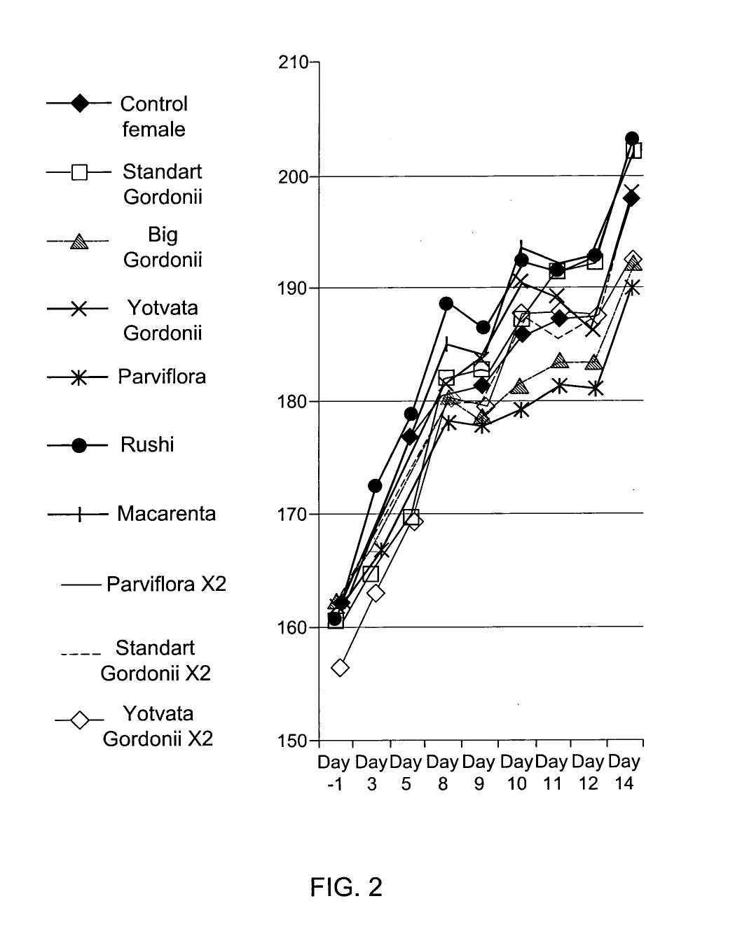

FIG. 2 shows that the body weight gain in female rats at days 8, 9, 10, 11, 12 and 14 was significantly lower in female rats who received Hoodia parviflora compared to rats who received other species of Hoodia.

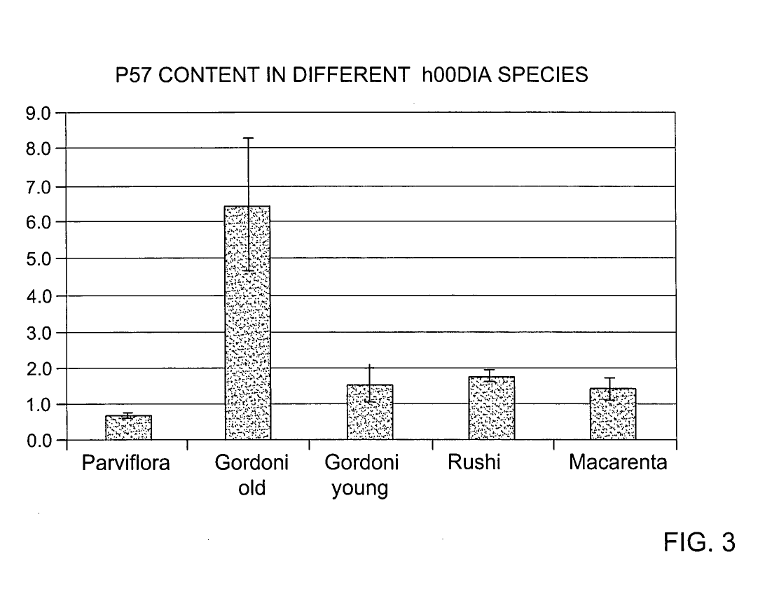

FIG. 3 shows that the P57 content in Hoodia parviflora is lower than that of other Hoodia species.

Therefore, even though the p57 content of Hoodia parviflora is lower than that in other species of Hoodia, and significantly lower than in Hoodia Gordonii, the Hoodia parviflora extract had a significantly higher food suppressant effect compared to the other Hoodia species.

### Example 3

Sap obtained from Hoodia Parviflora and Hoodia Gordonii was analyzed for chemical content. The results are provided in the tables below.

TABLE I
Ion Chromatography

| (mg/L) | Parviflora | Gordonii |
|---|---|---|
| NO₂ | <0.2 | <0.2 |
| Cl⁻ | 4976.6 | 5697.5 |
| Br⁻ | 3.3 | 5.8 |
| NO₃ | 20.2 | 41.7 |
| Fl⁻ | 13.8 | 7.6 |
| PO₄⁻³ | 297.3 | 195.3 |
| SO₄⁻² | 151.3 | 63.6 |

TABLE II
Inductively Coupled Plasma Spectrometry

| (mg/L) | Parviflora | Gordonii |
|---|---|---|
| Ag | <0.1 | <0.1 |
| Al | 1.5 | 2 |
| As | <0.5 | <0.5 |
| B | 3 | 3 |
| Ba | 0.05 | 0.105 |
| Be | <0.05 | <0.05 |
| Ca | 119 | 135 |
| Cd | <0.05 | <0.05 |
| Co | <0.05 | <0.05 |
| Cr | 0.06 | 0.06 |
| Cu | <0.05 | <0.05 |
| Fe | 19 | 7.1 |
| Hg | <0.3 | <0.3 |
| K | 220 | 117 |
| Li | 0.41 | 0.62 |
| Mg | 224 | 366 |
| Mn | 0.23 | 0.16 |
| Mo | <0.05 | <0.05 |
| Na | 2360 | 2384 |
| Ni | <0.2 | <0.2 |
| P | 37 | 44 |
| Pb | <0.25 | <0.25 |
| S | 1135 | 1461 |
| Sb | <0.5 | <0.5 |
| Sc | <0.5 | <0.5 |
| Si | <0.5 | <0.5 |
| Sn | <1.0 | <1.0 |
| Sr | 1.7 | 0.91 |
| Ti | <0.05 | <0.05 |
| V | <0.05 | <0.05 |
| Zn | 1 | 1.3 |

TABLE III
HPLC

| (mg/L) | Parviflora | Gordonii |
|---|---|---|
| tartaric acid | 71 | |
| Acetic acid | | 501.4 |
| malic acid | 117 | 143.3 |
| Succinic acid | 97.6 | 111 |
| citric acid | 19.1 | 17.8 |
| oxalic acid | 129.9 | 21.4 |
| fructose | 464.5 | 338.8 |
| glucose | 3083.8 | 5038.1 |
| sucrose | 1867.2 | 1029.1 |

TABLE IV
Gas chromatography/mass spectrometry (GC/MS)

| (area) | Parviflora | Gordonii |
|---|---|---|
| unknown a | 669757 | 142478 |
| unknown b | 380038 | |
| unknown c | 480005 | 340551 |
| unknown d | 433926 | 202960 |
| unknown e | 141626 | 191901 |
| unknown f | 375844 | 240440 |
| unknown g | 952862 | 573343 |
| 2(3H)-Furanone | 133196 | |
| Butanedioic acid | 558330 | 399331 |
| Trihydroxybutyric | 522623 | 317059 |
| acid | | |
| Xylonic acid | 157086 | |
| unknown 1 | | 85591 |
| unknown 2 | 157309 | |
| unknown 3 | | 151100 |
| Xylonic acid | | 169073 |
| unknown 4 | 1160190 | 806929 |
| unknown 5 | 456698 | 383243 |
| D-Fructose | 506327 | 296741 |
| unknown 6 | | 216258 |
| unknown 7 | 478320 | |
| Sorbose | 432752 | |
| unknown 8 | 157472 | |
| unknown 9 | | 88187 |
| D-Glucose | 7678156 | 3007974 |
| unknown 10 | 66180 | |
| unknown 11 | 145755 | 282284 |
| Germanicol | 8401421 | 3317826 |
| unknown 12 | | 110227 |
| unknown 13 | 436539 | 206503 |
| unknown 14 | | 90529 |
| unknown 15 | 188743 | |
| Inositol | | 343829 |
| unknown 16 | 148328 | |
| unknown 17 | | 486720 |
| unknown 18 | 210271 | |
| unknown 19 | | 1108783 |

It can be seen from the above results that although Hoodia Parviflora differs significantly from Hoodia Gordonii in its chemical content, it also has appetite suppressant activity which is even superior to that of Hoodia Gordonii.

### Example 4

The P57 content of liquid extracts of Hoodia Parviflora and Hoodia Gordonii were compared. The results are shown in Table V.

TABLE V
P57 (ug/ml) results for liquid

| Parviflora | Gordonii | pasteurized Gordonii |
|---|---|---|
| 265 | 2710 | 2000 |

It can be seen that although pasteurization of the extract of Hoodia Gordonii resulted in a large reduction in P57 content, the P57 content of Hoodia Parviflora remains significantly less than that of Hoodia Gordonii.

1.-22. (canceled)

23. An extract of Hoodia Parviflora.

24. The extract according to claim 23, wherein the extract is liquid.

25. The extract according to claim 23, wherein the extract is solid.

26. A composition comprising the extract of claim 23, or mixtures thereof.

27. A composition containing an appetite suppressant effective amount of an extract according to claim 23 or mixtures thereof.

28. The composition according to claim 26, wherein the composition is a pharmaceutical composition.

29. The composition according to claim 26, wherein the composition is edible.

30. The composition according to claim 29, wherein the edible composition is selected from the group consisting of a dietary supplement, a nutraceutical, a food additive, a food product, and a beverage.

31. The composition according to claim 30, wherein the food product is an ice-cream or a frozen-ice.

32. A frozen tablet containing the extract according to claim 23, or mixtures thereof.

33. A method of treating or preventing obesity comprising administering the composition of claim 26 to a subject in need thereof, suffering from, or at risk for, obesity.

34. A method of suppressing appetite in a subject comprising administering the composition of claim 26 to the subject.

35. The method according to claim 33, wherein the subject is a human or a non-human animal.

36. The method according to claim 34, wherein the subject is a human or a non-human animal.

37. A food additive comprising the extract of claim 23 or mixtures thereof.

38. A method of suppressing appetite in a subject comprising administering the food additive of claim 37 to the subject.

39. Ice cream comprising the extract of claim 23 or mixtures thereof.

40. Ice-cream according to claim 39, wherein the ice-cream is a popsicle.
